(19) United States
(12) Patent Application Publication
(10) Pub. No.: US 2024/0117542 A1
(43) Pub. Date: Apr. 11, 2024

(54) METHOD FOR APPLYING ELASTOMER ON BOTH SIDES OF A FABRIC LAYER

(71) Applicant: NTT NEW TEXTILE TECHNOLOGIES GMBH, BALINGEN (DE)

(72) Inventor: HANS BAUER, BALINGEN (DE)

(73) Assignee: NTT NEW TEXTILE TECHNOLOGIES GMBH, BALINGEN (DE)

(21) Appl. No.: 18/541,674

(22) Filed: Dec. 15, 2023

Related U.S. Application Data
(63) Continuation-in-part of application No. PCT/EP2023/025146, filed on Mar. 31, 2023.

(30) Foreign Application Priority Data
Apr. 29, 2022 (DE) 10 2022 110 520.2

(51) Int. Cl. D06B 11/00; D06Q 1/14

(52) U.S. Cl. CPC D06B 11/0056; D06Q 1/14; D10B 2501/00; D10B 2509/02

(57) ABSTRACT

In a method for the application of an elastomer to both sides of a fabric layer, the fabric layer is placed onto a resilient support and the elastomer is applied to the fabric layer in a flowable state under pressure exclusively from one side and the resilient support is compressed during the application of the elastomer by the pressure of the elastomer.

### CROSS-REFERENCE TO RELATED APPLICATIONS

This is a continuation-in-part application of pending international patent application PCT/EP2023/025146 filed Mar. 31, 2023 and claiming the priority of German patent application No. 10 2022 110 520.2 filed Apr. 29, 2022. Both the said international patent application PCT/EP2023/025146 and the said German patent application No. 10 2022 110 520.2 are incorporated herein by reference in their entireties as though fully set forth.

### BACKGROUND OF THE INVENTION

The invention resides in a method for applying elastomer on both sides of a fabric layer, for example a fabric layer of a piece of clothing.

In a method as known from EP 1 211 956 B2 for the manufacture of underwear such as a bra, underpants, sports pants and similar, an upper layer and a lower layer of the piece of underwear are interconnected in the edge area by the application of an elastomer adhesive which is applied by a screen printing process. The upper layer of the clothing piece is placed onto the lower layer and pressed on. The edge area of the piece of underwear is defined by the elastomer adhesive connection. Outside the adhesive connection, the layers are disposed loosely on top of one another.

DE 199 42 996 B4 discloses a method for the manufacture of a piece of underwear with a fabric support material of at least one layer on one side of which an elastomer adhesive is applied that is subsequently coated with a flocking material.

From the publication Joshi, M., Butola, B. S.: “Application technologies for coating lamination and finishing textiles.” In: Advances in the dyeing and finishing of technical textiles, Oxford: Woodhead Publishing, 2013 (Woodhead Publishing series in textiles)—ISBN 978-0-85709-433-9, it is known to extend a rubber belt around two rotating drums and to place onto the rubber belt a fabric layer which is coated by a screen printing machine. Because of its elasticity, the rubber belt can expand slightly between the drums and yield when the screen printing machine moves over the fabric layer.

EP 2 365 493 A1 discloses a method for the manufacture of a flexible PTC heating element. In this method a PTC paste, which may contain a polymer as an adhesive is pressed through a grid-like structure which is supported on a resilient surface, for example, a formed polyurethane support. The resilient surface forms a barrier for the PTC paste.

It is the object of the present invention to apply an elastomer on both sides of a fabric layer in a simple and efficient manner.

### SUMMARY OF THE INVENTION

The object is achieved by the method as defined in claim 1. With the method according to the invention a fabric layer can be coated on both sides with an elastomer. The fabric layer is used preferably for a clothing piece for example an underwear item or an upper wear clothing piece. The fabric layer may be used for example in connection with bras, slips, sport pants or T-shirts. It may also be used in connection with joint bandages for example knee and elbow bandages.

With the method elastomer is applied in a simple and efficient manner to both sides of a fabric layer. The material of the fabric layer has preferably an open structure and is for example, in the form of tulle or mesh. In a first step, the fabric layer is placed onto a resilient support. In the next step the elastomer is applied in a fluid state under pressure onto one side of the fabric layer. In this process the pressure is so selected that the resilient support on which the fabric layer is disposed is compressed during the application of the elastomer at least slightly and locally delimited.

As a result of the resiliency of the support on which the fabric layer is disposed, the elastomer, which is applied from the top under pressure, penetrates the fabric layer completely and reaches the opposite side of the fabric facing the resilient support. The pressure under which the elastomer is applied compresses the resilient support which facilitates the distribution of the elastomer within the fabric layer up to the point where the elastomer reaches the side facing the support.

It is sufficient that the elastomer is applied to the fabric layer exclusively from the top and under pressure for the application of the elastomer in a single process step. It is not necessary to apply additional elastomer in a second process step.

The pressure with which the elastomer is applied to the fabric layer compresses the preferably elastically resilient support partially. The resilient support is compressed by the pressure during application of the elastomer by at least 1%. If appropriate also a compression of at least 5%, at least 10%, at least 15%, at least 20% or even more is possible. In accordance therewith the pressure is adapted to the resiliency of the support.

The pressure with which the elastomer is applied to the fabric layer in a fluid state is further so selected that the resilient support is compressed maximally by 50%, for example maximally by 40% or 30%. The limitation of the pressure to a maximum value ensures a rapid execution of the process step and a uniform application of the elastomer.

After the application of the elastomer, in a following process step, in a still uncured state of the elastomer a flocking material may be applied to at least one side of the fabric layer. This can be done in that, after the application of the polymer, a flocking material is sprayed onto the elastomer before it is cured so that the flocking material is firmly attached to the elastomer after curing of the elastomer and connected to the fabric layer. The flocking material is skin friendly and improves the wearing comfort as it is humidity absorbent. The flocking material consists preferably of natural fibers, synthetic fibers or half-synthetic fibers.

Also, both sides of the fabric layer may be provided with a flocking material. To this end, the fabric layer is removed from the resilient support after the flocking material has been applied to the top side and then the flocking material is applied to the bottom side.

The elastomer can be applied to the fabric layer evenly over an area or in a certain pattern.

The resilient support has a planar straight-lined surface, onto which the fabric layer is placed. It may be expedient that the resilient surface is uniformly compressed when the elastomer is applied. The resilient support is preferably in the form of a square i.e., a square block.

As a resilient support onto which the fabric layer is placed a foam material block support is used. Generally, supports may be used which are manufactured from an elastically resilient material.

On the resilient support a low friction additional layer is deposited onto which the fabric layer is placed. The low-friction additional layer prevents the elastomer which passes through the fabric layer and extends to the resilient support from sticking. As an additional layer for example a Teflon layer may be used.

The low friction additional layer may also be in the form of a separate component which is disposed on the resilient support and may be fixed thereto. Alternatively, the low-friction additional layer may be firmly connected to the support and forms the top side of the support onto which the fabric layer is placed.

The method according to the invention where elastomer is applied to both sides of a fabric layer includes the following steps:
the fabric layer is placed onto a resilient support,
the elastomer is applied to the fabric layer in a liquid state under pressure exclusively from one side and the resilient support is compressed by the pressure of the elastomer during the application of the elastomer.
as resilient support onto which the fabric layer is placed a foam material layer is used,
on the foam material layer a low-friction additional layer is disposed,
the elastomer applied from the top under pressure penetrates the fabric layer completely and reaches the opposite side of the fabric layer facing the resilient support.

As a result, the invention resides in a method for applying an elastomer to both sides of the fabric layer wherein the fabric layer is placed onto a resilient support and the elastomer is applied in a flowable state under pressure onto the fabric layer wherein the elastomer is applied exclusively from one side of the fabric layer and the resilient support is compressed during the application of the elastomer under the pressure of the elastomer. As resilient support onto which the fabric layer is placed a foam material layer is used which is provided with a low-friction additional layer. The elastomer applied from above under pressure penetrates the fabric layer completely and reaches the opposite side of the fabric layer facing the resilient support.

In a further advantageous embodiment, the elastomer is applied to the fabric layer by a screen printing process. Herein a screen printer screen is placed onto the fabric layer and elastomer in a liquid state is applied to the screen printer screen. Then a screen printing machine is moved over the screen printer screen which exerts a pressure onto the elastomer and presses the elastomer through the screen printer screen and into the fabric layer disposed below the screen printer screen. The screen printer screen may have uniformly distributed openings or it may be in the form of a template which has openings arranged in a particular pattern through which the elastomer is pressed by the screen printing machine so as to form in the fabric layer a corresponding pattern.

As elastomer for example a thermoplastic elastomer is used which when heated softens or becomes liquid and hardens upon cooling. In a heated state the elastomer can be applied via a nozzle. When cooling the elastomer hardens and forms a firm connection with the fabric layer and, additionally protects the screen printer wiper and secures the fabric layer. In the hardened state the elastomer still has an elastic behavior. As elastomer a silicon is used.

It may be sufficient to use a single fabric layer onto which elastomer is deposited in a liquid state and under pressure so that the elastomer spreads out up to the opposite side. In another advantageous embodiment several fabric layers, preferably two fabric layers can be placed directly on top of each other wherein elastomer in a liquid state is applied under pressure to the uppermost fabric layer and spreads up to the opposite side of the fabric layer and through the underlying fabric layer until the elastomer reaches the lower side of the underlying fabric layer.

The invention furthermore may be used in connection with a clothing piece or bandage which is produced in accordance with the above described method. The clothing piece or the bandage can be produced either exclusively from such a fabric layer or several such fabric layers. Additionally to such a fabric layer or several such fabric layers it may include fabric layers of different set-ups.

Further advantages and expedient embodiments are apparent from the claims and the following description with reference to the accompanying drawings.

### BRIEF DESCRIPTION OF THE DRAWINGS

It is shown:

FIG. 1 is a cross-sectional view of an arrangement applying elastomer to both sides of a fabric layer shown at the beginning of the process,

FIG. 2 is a view like FIG. 1 but at an advanced time in the application process.

In the figures the same components are designated by the same reference numerals.

### DETAILED DESCRIPTION OF THE INVENTION

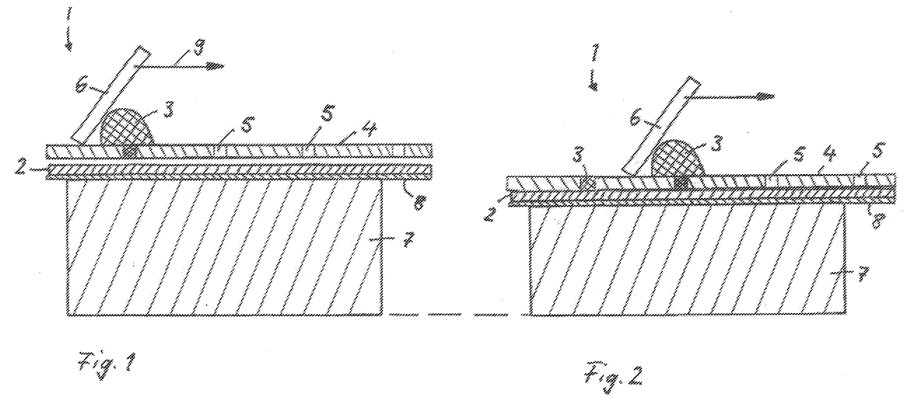

FIG. 1 shows an arrangement 1 for applying elastomer 3 to the two opposite sides of a fabric layer 2. The arrangement 1 is a screen printing arrangement whereby the elastomer 3 is applied in a flow-capable, that is, uncured state with the aid of a screen printer screen 4 and a screen printer wiper 6 from above onto the fabric layer 2. Herein the elastomer 3 is deposited on the screen printer screen 4 and the screen printer wiper 6 is moved in a horizontal direction over the screen printer screen 4 as indicated by arrow 9. The screen printer wiper 6 is arranged inclined with respect to a horizontal plane with the angle between the screen printer wiper 6 and the horizontal plane being in the range of 45° to 60°, so that the elastomer 3 is moved on one hand in a horizontal direction over the screen printer screen 4 and on the other hand, a downward directed vertical force is effective on the elastomer 3 which presses the elastomer under pressure through the screen printer screen.

The fabric layer 2 is supported on a resilient support 7 in the form of a foamed material layer which is an elastically resilient foam material block 7. On the foam material block 7 a low-friction additional layer 8 is disposed which may be for example a Teflon layer 8 and on which the fabric layer 2 to be treated is disposed. As a result of the resiliency, the foam material block 7 can be compressed when, upon a movement of the screen printer 6 in the direction of the arrow 9, a vertical downwardly directed pressure force component is applied to the elastomer 3.

When the screen printer wiper 6 is moved in the direction of the arrow 9 over the screen printer screen 4 the elastomer 3, which is disposed on the screen printer screen 4, is pressed through the openings 5 in the screen printer screen 4 downwardly toward the underlying fabric layer 2 and is distributed through the pores of the fabric layer 2. From the upper side of the fabric layer 2 the elastomer passes through the fabric layer 2 to the lower side of the fabric layer 2 which faces the foam material block 7 so that the upper side of the fabric layer 2 as well as the lower side of the fabric layer 2 are soaked by the elastomer 3.

As a result of the elastically resilient behavior of the foam material block 7, the foam material block 7 is compressed by a movement of the screen printer wiper 6 over the screen printer screen 4 and the downwardly directed vertical force generated thereby as apparent from a comparison of FIG. 1 and FIG. 2. The parameters of the arrangement 1 with regard to the inclination of the screen printer wiper 6, the size of the openings 5 in the screen printer screen 4, the fabric of the fabric layer 2, the elasticity of the foam material block 7 and the viscosity of the elastomer 3 and also the process parameters with regard to the movement speed of the screen printer wiper 6 can be tuned so that the foam material block 7 is compressed to the desired degree. The compression of the foam material block 7 is for example in the range of between 1% and 50% with respect to the height of the foam material block 7 under no-load conditions.

The vertical compression of the foam material block 7 during movement of the screen printer 7 according to arrow 9 supports and improves the distribution of the elastomer 3 from the upper side to the lower side of the fabric layer 2. Under the pressure of the elastomer 3 the foam material block 7 yields downwardly so that the elastomer 3 can slightly progress from the lower side of the fabric layer 2. The elastomer which is present below the lower side of the fabric layer 2 may be pushed back into the fabric layer 2 by a spring reaction movement of the foam material block 7. In any case it is ensured however that the elastomer reaches the lower side of the fabric layer 2.

Depending on the version of the screen printer screen 4, different elastomer patterns can be generated on the upper side and the lower side of the fabric layer 2. Areal-like, line-like or dot-like elastomer depositions are possible.

After completion of the screen printing procedure the fabric layer 2 may be flocked on one side or both sides thereof. To this end, a flocking material is applied to the upper side of the fabric layer 2 before the elastomer is cured. A flocking material may also be applied to the lower side of the fabric layer 2. In this case the fabric layer 2 may be turned over in the arrangement 1 so that the originally lower side faces upwardly and flocking material can be applied thereto. Upon curing of the elastomer 3 the flocking material is firmly connected to the one or both sides of the fabric layer 2.

### LISTING OF REFERENCE NUMBERS

1 screen printing arrangement
2 fabric layer
3 elastomer
4 screen printer screen
5 opening
6 screen printer wiper
7 resilient support/foam material layer
8 additional Teflon layer
9 arrow

What is claimed is:

1. A method for applying elastomer (3) to both sides of a fabric layer (2) for a clothing piece or a joint bandage in which the fabric layer (2) is placed on a resilient support (7) and the elastomer (3) is applied in a flowable state under pressure to the fabric layer (2), wherein the elastomer (3) is applied to the fabric layer (2) exclusively from one side thereof and, during the application of the elastomer under pressure, the resilient support (7) is compressed, characterized in that,
the resilient support (7) onto which the fabric layer (2) is placed is a foam material layer (7) provided with a low friction additional layer (8) and that the elastomer (3), which is applied from above under pressure, completely penetrates the fabric layer (2) and reaches the opposite side of the fabric layer 2) facing the resilient support (7), wherein as elastomer (3) a silicon is used and wherein the resilient support (7) is compressed by the pressure during the application of the elastomer (3) by at least 1% and maximally 50%.

2. The method according to claim 1, characterized in that,
after the application of the elastomer (3) before curing of the elastomer (3) a flocking material is applied to at least one side of the fabric layer (2), preferably to both sides of the fabric layer (2).

3. The method according to claim 1, characterized in that,
the additional layer (8) is a Teflon layer.

4. The method according to claim 1, characterized in that,
the elastomer (3) is applied to the fabric layer (2) by a screen printing process.

5. The method according to claim 1, characterized in that,
elastomer (3) is applied under pressure in a flowable state to exactly one fabric layer (2).

6. The method according to claim 1, characterized in that,
two fabric layers (2) are disposed directly on top of one another and elastomer (3) is applied in a flowable state under pressure onto the upper fabric layer (2).

7. A clothing piece or bandage with a fabric layer (2) which is produced according to the method of claim 1.

8. The method according to claim 2, characterized in that,
the flocking material consists of natural fibers, synthetic fibers or half-synthetic fibers.
